(19) United States
(12) Patent Application Publication
(10) Pub. No.: US 2021/0060305 A1
(43) Pub. Date: Mar. 4, 2021

(54) Fenestration Over the Needle Catheter

(71) Applicant: Avent, Inc., Alpharetta, GA (US)

(72) Inventors: Kunal M. Amin, La Mirada, CA (US); Angela M. Mulcahy, South Lyon, MI (US)

(21) Appl. No.: 16/957,759

(22) PCT Filed: Dec. 28, 2017
(86) PCT No.: PCT/US2017/068709

§ 371 Date: Jun. 25, 2020

(51) Int. Cl. A61M 25/06; A61M 25/00

(52) U.S. Cl. CPC A61M 25/0606; A61M 2202/048; A61M 25/0631; A61M 25/0097

(57) ABSTRACT

A fenestration over-the-needle catheter assembly which includes a catheter with a catheter body. The catheter body has a projection which extends in an axial direction from the distal end of the catheter. The projection may have a point of connection to the catheter body such that the projection can be transitioned into a closed position, covering an opening in the distal end of the catheter. The projection may be configured to interact with a needle which has been inserted into the lumen and extends through the opening in the distal end, such that the projection is in an open position when the needle is extended through the opening in the distal end and is in a closed position when the needle is withdrawn into the lumen or removed from the catheter.

### BACKGROUND

Prior to, during, or after performing a surgical operation on a part of the body, it may be desirable to perform a nerve block to anesthetize the nerve bundles in the part of the body proximate to or at where the surgical incision will occur. Often, a catheter-based infusion system is utilized to block the nerve bundles at the incision, after surgery to provide a continuous, low flow rate of the anesthetic over a period of time (e.g., 2-5 days following surgery) for post-operative pain management.

As catheters are generally made of a flexible plastic material, a needle/introducer sheath is typically used to insert the catheter within a patient. For example, certain catheters, generally referred to as introducer needle/sheath systems, consist of a needle, peel away sheath and catheter for placement within the anatomy. Thus, the catheter can be inserted through the sheath after the needle/sheath is positioned at a targeted site and the needle is removed. Typically, such introducer needle/sheath systems require extra time for placement and leakage around the catheter is common because the needle insertion site is larger than the catheter. Another problem with this approach is that the catheter can easily be pulled out inadvertently, and pieces of the pull away sheath can also break in the patient.

Another type of catheter, generally referred to as an over-the-needle (OTN) catheter, includes a catheter coaxially mounted onto a needle. In this type of catheter, the catheter and the needle may be inserted into a patient together. Once the catheter and the needle are located at the targeted site, the needle can be removed, leaving the catheter in place. Thus, OTN catheters can be purposely directed to a targeted site within a patient without the need to thread a catheter through a sheath after the needle has been placed and removed. Accordingly, OTN catheters have gained increased attention in regard to delivering anesthetic medication, for example, for the purposes of performing a nerve block. However, a problem with OTN catheters is that the tip of the catheter generally must remain open as it is formed such that a tip of the needle extends through the opening so that an incision can be made by the needle. This has limited the use of OTN catheters in surgical settings as the open end of the catheter makes it extremely difficult to provide an even distribution of flow of the anesthetic to the target area.

Therefore, an improved OTN catheter that allows for controlled or even distribution of a drug (e.g., anesthetic) flow would be advantageous. Additionally, an improved OTN catheter that allows for easier placement of the catheter would be useful. It would also be beneficial for an improved OTN catheter to require less time for placement of the catheter. It would be a further advantage to decrease leakage at the insertion site of an OTN catheter. Moreover, an improved OTN catheter that covers a larger area of nerves would be advantageous.

### SUMMARY

In general, the present disclosure is directed to an over the needle (OTN) catheter assembly. The OTN catheter assembly may have a proximal end and a distal end, a body that extends from the proximal end to the distal end in an axial direction, wherein the body defines a lumen extending from the proximal end to the distal end. The body may have at least one aperture and a projection that extends axially from the distal end of the body when the projection is in an open position. The projection may be sized and connected to the body such that the projection closes an opening in the distal end of the catheter when the projection is transitioned into a closed position. The OTN catheter assembly may also comprise a needle that may be releasably configured within the lumen of the catheter. The needle may extend beyond the distal end of the catheter in a first position and may be withdrawn into the lumen or may be removed from the catheter in a second position.

In a second embodiment, the OTN catheter assembly may further comprise a hub which can be fixedly or releasably secured to the proximal end of the catheter.

In another embodiment, the projection may be attached to the body via a heat treatment, mechanical chemical treatment, or an adhesive. The projection may additionally or alternatively have an axial length from a point of connection with the body to a distal end of the projection that is at least as long as an outer diameter of the catheter. In yet a further embodiment, the projection may be configured to transition into the closed position when the needle is withdrawn into the lumen. In another embodiment, the projection may be configured to close the distal end such that the distal end of the catheter is beveled, flat, or blunt when the projection is in the closed position.

In an alternative embodiment, the OTN catheter assembly may generally comprise at least about 2 apertures. In another embodiment, each aperture may have a diameter from about 100 micrometers to about 1 millimeter. In yet a further embodiment, the apertures may be radially spaced apart around the body of the catheter or may be axially spaced apart around the body of the catheter or may be spaced both radially and axially around the body of the catheter.

Alternatively or additionally, the catheter of the OTN catheter assembly may be comprised of a polyester, a polyurethane, a polyamide, a polyolefin, or a combination thereof. In another embodiment, the catheter may include a reinforcing material or a flexible material. In an additional embodiment, a portion of the distal end opposite the projection may be configured to releasably or permanently attach to the projection when the projection is in the closed position.

The present disclosure may also be generally directed to an over the needle (OTN) catheter assembly. The OTN catheter assembly may have a proximal end and a distal end, a body that extends from the proximal end to the distal end in an axial direction, wherein the body defines a lumen extending from the proximal end to the distal end. The body may have at least one aperture and a projection that extends axially from the distal end of the body when the projection is in an open position. The projection may be sized and connected to the body such that the projection closes an opening in the distal end of the catheter when the projection is transitioned into a closed position. The lumen may have a diameter larger than a diameter of a needle, wherein the needle extends beyond the distal end of the catheter when the needle is fully inserted through the opening in the distal end of the catheter. The projection may be connected to the body such that the projection is in the open position when the needle is fully inserted through the opening in the distal end of the catheter and transitions into the closed position when the needle is retracted from the lumen such that the needle does not extend past the distal end of the catheter.

Additionally, the present disclosure may generally be directed to a method for administering a bolus or continuous flow of a drug or anesthetic to a target area of a patient comprising piercing a surface of skin with an OTN catheter assembly. The OTN catheter assembly may have a proximal end and a distal end, a body that extends from the proximal end to the distal end in an axial direction, wherein the body defines a lumen extending from the proximal end to the distal end. The body may have at least one aperture and a projection that extends axially from the distal end of the body when the projection is in an open position. The projection may be sized and connected to the body such that the projection closes an opening in the distal end of the catheter when the projection is transitioned into a closed position. The OTN catheter assembly may also comprise a needle that may be releasably configured within the lumen of the catheter. The needle may extend beyond the distal end of the catheter in a first position and may be withdrawn into the lumen or may be removed from the catheter in a second position. The distal end of the OTN catheter assembly may be positioned in a target area of a patient, and the needle may be withdrawn into the lumen at least to a point of connection between the body and the projection, or the needle maybe withdrawn completely such that the projection transitions into the closed position. A bolus or continuous flow of a drug or anesthetic may then be administered to the target area.

In an alternative embodiment, the catheter may be repositioned after the needle has been at least partially withdrawn, or the needle may be withdrawn prior to reaching the target area. Additionally or alternatively, the OTN catheter assembly may be positioned near a nerve or nerve bundle. In a further embodiment, the at least one aperture is sized such that a drug or anesthetic may be administered at a rate of about 0.01 ml/hr to about 35 mL/hr. In yet another embodiment, the opening in the distal end may be completely closed by the projection such that no drug or anesthetic is administered from the opening in the distal end and a drug or anesthetic is only administered from the at least one aperture.

### BRIEF DESCRIPTION OF THE DRAWINGS

The invention will now be described, by way of example, with reference to the accompanying drawings, in which:

FIG. 1 illustrates a side perspective view of the OTN catheter assembly of an embodiment according to the present disclosure;

FIG. 2 illustrates a side perspective view of the OTN catheter assembly of FIG. 1 showing the embodiment with a closed distal end;

FIG. 3 illustrates a side perspective view of the OTN catheter assembly of another embodiment according to the present disclosure;

FIG. 4 illustrates a side perspective view of the OTN catheter assembly of FIG. 3 showing the embodiment with a closed distal end;

FIG. 5 illustrates a side perspective view of the OTN catheter assembly of an additional embodiment according to the present disclosure;

FIG. 6 illustrates a side perspective view of the OTN catheter assembly of FIG. 5 showing the embodiment with a closed distal end;

FIG. 7A illustrates a side perspective view of the OTN catheter assembly of yet another embodiment according to the present disclosure;

FIG. 7B illustrates a side perspective view of the OTN catheter assembly of FIG. 7A showing the embodiment with a partially closed distal end;

FIG. 7C illustrates a side perspective view of the OTN catheter assembly of FIG. 7A showing the embodiment with a closed distal end;

FIG. 7D illustrates a side perspective view of the OTN catheter assembly of FIG. 7A showing the embodiment with a needle fully removed from the lumen;

FIG. 8A illustrates a side perspective view of the OTN catheter assembly of a further embodiment according to the present disclosure;

FIG. 8B illustrates a side perspective view of the OTN catheter assembly of FIG. 8A showing the embodiment with a closed distal end;

FIG. 9 illustrates a close up side perspective view of the distal end of an OTN catheter assembly according to the present disclosure;

FIGS. 10A and 10B illustrate cross-sectional views of embodiments of the OTN catheter assembly according to the present disclosure.

FIGS. 11A and 11B illustrate cross-sectional views of embodiments of the OTN catheter assembly according to the present disclosure.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

The positional terms “proximal” and “distal” are used herein to orient the various components relative to each other. “Distal” refers to the direction that would be closest to a catheter insertion site in a patient, and “proximal” refers to the opposite direction. The positional terms “axial” and “transverse” are also used herein to orient the various components relative to each other. “Axial” refers to the direction generally extending from the distal end to the proximal end, and “transverse” refers to the direction extending through the catheter and generally perpendicular to the catheter body.

Generally, the present disclosure is directed to an OTN catheter assembly that may be used to administer a drug (e.g. anesthesia) during or after a surgical procedure or during or after an incisional nerve block procedure. An OTN catheter assembly of the present disclosure may comprise a catheter with a body that may have at least one aperture and a projection that extends axially from the distal end of the body when the projection is in an open position. The projection may be sized and connected to the body such that the projection may close an opening in the distal end of the catheter when the projection is transitioned into a closed potion. The OTN catheter assembly may further comprise a needle releasably configured within a lumen of the catheter such that the needle extends beyond the distal end of the catheter in a first position and is withdrawn into the lumen or is removed from the catheter in a second position.

A benefit of using an OTN catheter is that it eliminates the step of feeding the catheter through the needle, saving time and also lessening the chance of moving the catheter away from the target area. However, OTN catheters have not been used in and after surgical and nerve block procedures in particular, as the catheter has an opening in the distal end where the needle protrudes through the catheter in order to form the incision. This makes it extremely difficult to control an amount or a rate of administration of a drug or anesthesia. Therefore, by providing an OTN catheter with a projection that may close an open distal end of a catheter, an OTN catheter may be used in circumstances where greater control of the amount or rate of a drug or anesthesia is needed.

Accordingly, the OTN catheter assembly may generally include a catheter having a proximal end and a distal end and a body that extends in an axial direction from the proximal end to the distal end. The distance between the proximal and distal ends of the catheter may generally correspond to the length of a needle used with the catheter, or the catheter may have a greater or lesser length than the length of the needle. For instance, the length of the catheter or needle may be greater than about 2 centimeters, such as about 5 centimeters or greater, such as about 7 centimeters or greater, such as about 10 centimeters or greater, such as about 12 centimeters or greater, such as about 15 centimeters or greater, such as about 20 centimeters or greater, such as about 25 centimeters or greater, such as about 30 centimeters or greater, such as about 35 centimeters or greater, such as about 40 centimeters or greater, such as about 45 centimeters or greater, such as about 50 centimeters or greater, such as about 55 centimeters or greater, such as about 60 centimeters or greater, such as less than about 80 centimeters, such as about 75 centimeters or less, such as about 70 centimeters or less, such as about 65 centimeters or less, such as about 60 centimeters or less, such as about 55 centimeters or less, such as about 50 centimeters or less, such as about 45 centimeters or less, such as about 40 centimeters or less, such as about 35 centimeters or less, such as about 30 centimeters or less, such as about 25 centimeters or less, such as about 20 centimeters or less, such as about 15 centimeters or less in length. However, the catheter and/or needle may have a larger or smaller length in other embodiments. The catheter may include a piezoelectric or echogenic component as known in the art that may be configured within the body material or inside of the catheter, or alternatively may include no positioning aid, or another positioning aid known in the art.

The catheter may generally have a shape and size as generally known in the art such as described by the French gauge scale. Alternatively, the catheter may be tapered towards the distal end. In one embodiment, the catheter may be tapered such that the lumen of the catheter is also tapered, or, alternatively, only the exterior of the body may be tapered such that the outer diameter of the catheter is tapered without changing the diameter of the lumen which corresponds to the interior diameter of the catheter. The outer diameter of the lumen may be defined by the distance between opposing exterior edges of the catheter body, whereas the interior diameter or the lumen diameter may be defined by the distance between opposing interior edges of the catheter body. The lumen may have a length and diameter generally corresponding to the diameter and length of a needle that may be used with the catheter, or may have a smaller or larger length or larger diameter than a needle that may be used with the catheter. Regardless of the length and/or outer diameter of the catheter, the lumen may have a diameter larger than a diameter of a needle used with the OTN catheter assembly of the present disclosure. Alternatively, it may be desired that the catheter lumen have a relatively large diameter while maintaining the same outer diameter size for the catheter itself, thus, the catheter may have a thinner body, defined as the distance between the exterior diameter of the catheter body and the interior diameter of the catheter. In yet a further embodiment, it may be desirable to have a catheter body that is relatively thick, such that an outer diameter of the catheter may be maintained while the diameter of the lumen is decreased.

The catheter body of the present disclosure may also generally have an opening in the distal end. The opening may generally have the same shape and diameter as the shape and diameter of the catheter lumen. Alternatively, the opening in the distal end may have a smaller diameter than that of the catheter lumen. For example, the projection may extend into the opening from the catheter body, thus decreasing the diameter of the opening in the distal end. As an additional example, the diameter of the opening in the distal end may be smaller than the diameter of the catheter lumen and the projection may be made from a flexible material that may expand when subjected to a force, such as the introduction of a needle into the lumen, so as to form a releasable seal with the needle. Alternatively, the opening in the distal end may be larger than the diameter of the catheter lumen.

The catheter body may have a projection that extends in an axial direction from the distal end of the body when the projection is in an open position. The projection may generally be sized such that the projection may close an open distal end of the catheter when the projection is transitioned into a closed position. The projection may have an axial length from the point of connection with the body to the distal end of the projection that is at least as long as the outer diameter of the catheter. Alternatively, the projection may have an axial length that is shorter than an outer diameter of the catheter, such as an embodiment wherein the opposite side of the catheter body tapers towards the lumen or where the thickness of the catheter body is quite large such that the axial length of the projection is only as long as the diameter of the lumen. Alternatively, the projection may have an axial length that is longer than the outer diameter of the catheter.

The projection may be configured so that when the projection is transitioned into a closed position, an angle formed between the projection and the transverse direction is also closed, wherein the angle to be closed may range from about 1° to about 90°, where the angle to be closed may be generally shown in the Figures as ⊖₁. Of course the angle may be greater than about 90° depending upon the initial attachment to the catheter body, or it may be less. The degree or amount of closing may also depend on the shape and position of the termination of the portion of the open distal end opposite the projection. The portion of the open distal end opposite the projection may be tapered towards the needle such that the outer diameter of the catheter at the distal end may be smaller than an outer diameter at a proximal end of the catheter, or may be substantially consistent throughout the length of the catheter body. The portion of the open distal end opposite the projection may also have an interior surface that is generally planar with the catheter body and an exterior surface which may to taper towards the interior surface and/or towards the needle.

In an embodiment where the portion of the open distal end of the catheter opposite the projection has a taper, either in whole or in part, the taper may be fairly steep, such as at an angle of greater than about 45°, such as greater than about 50°, such as greater than about 55°, such as greater than about 60°, such as greater than about 70° where the degree of taper is the angle ⊖₂ measured between the plane of the exterior surface of the catheter body P and the tapered portion of the catheter body as shown in FIG. 1 for example. In an embodiment with a fairly steep taper, the tapered portion of the catheter body may be fairly short.

In an alternative embodiment, the tapered portion of the portion of the open distal end opposite the projection may be tapered at a fairly shallow angle, such as less than about 45°, such as less than about 40°, such as less than about 35°, such as less than about 30°, such as less than about 20° where the degree of taper is the angle ⊖₂ measured between the plane of the exterior surface of the catheter body P and the tapered portion of the catheter body as shown in FIG. 1 for example. In such an embodiment where the tapered portion of the catheter body is tapered at a fairly shallow angle, the tapered portion of the catheter body may be fairly long.

Further, the projection may extend linearly from the distal end of the catheter body or may have a curve or angle, such that the projection is curved or angled towards the catheter lumen. In a further embodiment, the projection may be formed from a deformable or flexible material such that the projection is generally linear but may be released into a curved or angled shape when a needle is removed from the lumen.

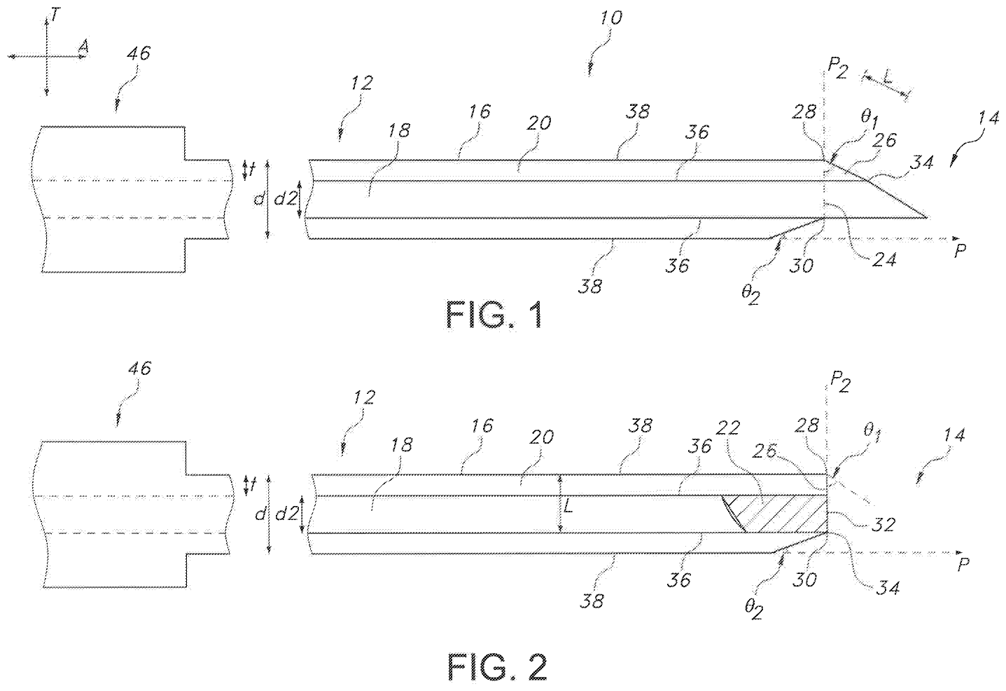

The shape of the projection as well as the degree of transition may be selected based upon the desired shape of the distal end when the projection is in a closed position. For example, in certain circumstances, it may be desirable to have a closed distal end with a slightly pointed shape, such as if additional positioning may be necessary, which may be generally shown in FIG. 6. Additionally, a beveled shape or flat shape may be selected to minimize damage to surrounding tissue, such as generally shown in FIGS. 2, 4, and 7. By adjusting the taper, shape, and/or degree of transition of the projection, the closed distal end may be formed from any number of shapes.

The body of the catheter may be comprised of various materials including polyesters, polyurethanes, polyamides, polyolefins, including polyethylene and polypropylene, and any copolymers thereof, such as nylon, polyester elastomer, polyether/block polyamide, and/or tetrofluorethylenes. Moreover, the body of the catheter may be fairly thick, wherein the thickness of the body is defined as the distance between the exterior diameter of the catheter body and the diameter of the catheter lumen and as generally shown by reference character “t”. For instance, the thickness of the body t may be greater than about 500 micrometers, such as about 600 micrometers or greater, such as about 700 micrometers or greater, such as about 800 micrometers or greater, such as about 900 micrometers or greater, such as about 1000 micrometers or greater, such as about 1100 micrometers or greater, such as about 1200 micrometers or greater, such as about 1300 micrometers or greater, such as about 1600 micrometers or less, such as about 1500 micrometers or less, such as about 1400 micrometers or less, such as about 1300 micrometers or less, such as about 1200 micrometers or less, such as about 1100 micrometers or less, such as a thickness of about 1000 micrometers or less. The body material and thickness may be selected based upon the target location of the catheter, the likelihood of positioning the catheter after removal of the needle, and additional considerations. For example, a thicker body or a stronger material may be selected if the catheter may need to be repositioned after removal of the needle, or a thinner or weaker material may be selected so as to decrease bulk and potentially increase comfort in areas where the catheter is less likely to need to be repositioned.

The projection and the point of connection of the projection to the body may be comprised of the same material as the catheter body or they may be formed from a separate material. In one embodiment, the projection, the catheter body, and the point of connection may all be comprised of a separate material. In yet a further embodiment, the body, projection, and point of connection may all be comprised of the same material, but the point of connection of the projection may be heat treated, or undergo a similar process, such that the point of connection is formed so that the projection may transition into a closed position. In yet a further embodiment, the body, point of connection, and projection may all be formed from one material or separate materials but at least one may include an additional reinforcing material to harden the material or raise the durometer, or alternatively have an embedded metal or metal alloy therein, or a flexible material, or a combination of a flexible material and reinforcing material. In yet an additional embodiment the body, point of connection, and the projection may all include an additional reinforcing material, a flexible material, or a combination of a reinforcing material or a flexible material.

The catheter body may further comprise at least one aperture. While there may be more or less apertures, the catheter body may generally contain at least 1 aperture, such as at least about 2, such as at least about 3, such as at least about 4, such as at least about 5, such as at least about 7 such as at least about 10 apertures. The catheter body may also comprise less than about 20, such as less than about 18, such as less than about 15, such as less than about 13 apertures.

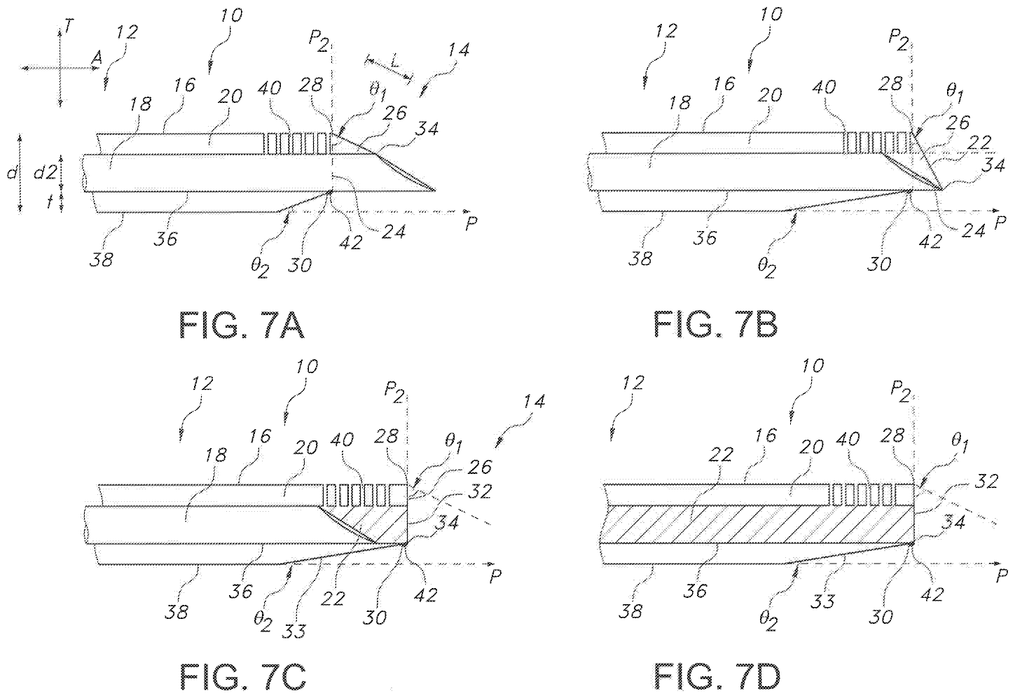

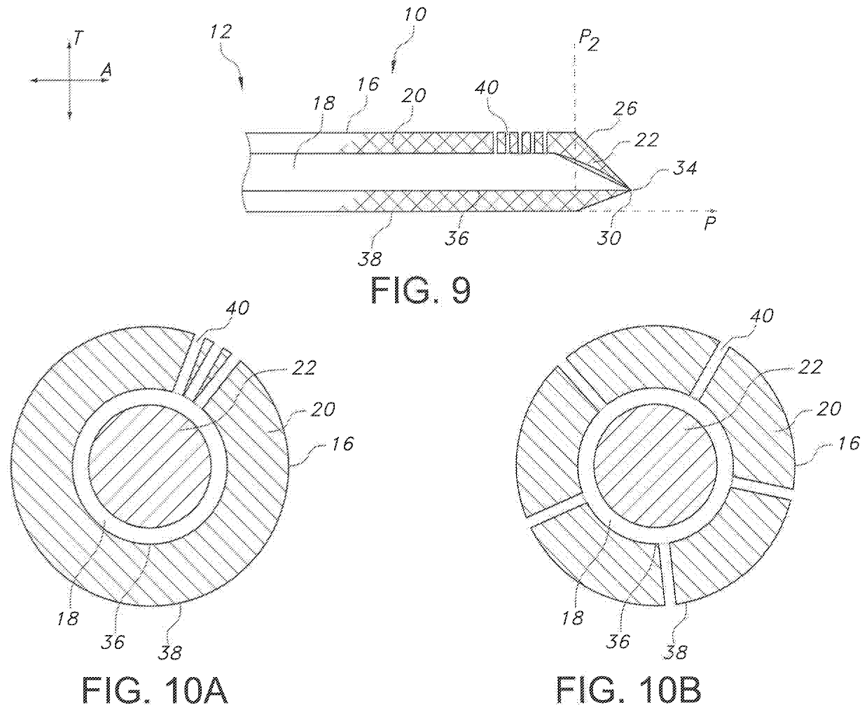

The apertures may be spaced apart radially as generally shown in FIG. 10B or grouped in a similar radial plane such as generally shown in FIG. 10A, axially as generally shown in FIGS. 7 and 9, or both radially and axially along the catheter body. In one embodiment, the apertures may be generally located adjacent to the distal end, such as within about 50 millimeters (mm) from the distal end, such as within about 45 mm, such as within about 40 mm, such as within about 35 mm, such as within about 30 mm, such as within about 25 mm, such as within about 20 mm, such as within about 15 mm, such as within about 10 mm of the distal end. In another embodiment, the apertures may be evenly or randomly spaced radially, axially, or a combination of radially and axially along the entirety of the length of the catheter body

The apertures may have a diameter such as about 1 millimeter or less, such as about 900 micrometers or less, such as about 800 micrometers or less, such as about 700 micrometers or less, such as about 600 micrometers or less, such as about 500 micrometers or less, such as about 400 micrometers or less, such as about 300 micrometers or less, such as about 100 micrometers or greater, such as about 200 micrometers or greater, such as about 300 micrometers or greater, such as about 400 micrometers or greater, such as about 500 micrometers or greater, such as about 600 micrometers or greater in diameter. In a further embodiment, some of the apertures may have a fairly large diameter and some of the apertures may have a fairly small diameter. In an embodiment with apertures that have a mixture of diameters, the ratio of the diameter types may be fairly even, or in an alternate embodiment there may be more large diameter apertures, such as twice as many large, such as three times as many large, such as five times as many large diameter apertures. Alternatively, there may be more small diameter apertures such as twice as many small, such as three times as many small, such as five times as many small diameter apertures.

The arrangement of the apertures, number of apertures, and diameter of the apertures may be selected based upon the target area, the type of drug or anesthesia to be delivered, or the desired rate of delivery, to name a few considerations. For example, a larger number of apertures or apertures with a larger diameter, or both, may be selected when a greater rate of delivery is desired. Alternatively, arrangements of apertures may be selected based upon the location and orientation of the target area. For example, if a small target area is desired, all of the apertures may be located fairly close together either radially or axially. In an additional embodiment for example, if a target area may only be located on one side of a catheter, then the apertures may mainly be located on a single side of the catheter body such that the apertures are all located on generally the same side, area, or radius. Alternatively, if a more central location is chosen for the catheter, the apertures may extend radially around the catheter body such that an aperture is located on several different radii around the catheter body.

In one embodiment, the rate of delivery of a drug or anesthetic using a catheter assembly according to the present disclosure may be at least about 0.01 mL/hr, such as about 0.05 milliliters(mL)/hr or greater, such as about 0.1 mL/hr or greater, such as about 0.5 mL/hr or greater, such as about 1 mL/hr or greater, such as about 3 mL/hr or greater, such as about 5 mL/hr or greater, such as about 7.5 mL/hr or greater, such as about 10 mL/hr or greater, such as about 12.5 mL/hr or greater, such as about 15 mL/hr or greater, such as about 17.5 mL/hr or greater, such as about 20 mL/hr or greater, such as about 22.5 mL/hr or greater, such as about 25 mL/hr or greater, such as about 27.5 mL/hr or greater, such as about 30 mL/hr or greater, such as about 35 mL/hr or greater, such as about 40 mL/hr or less, such as about 37.5 mL/hr or less, such as about 35 mL/hr or less, such as about 32.5 mL/hr or less, such as about 30 mL/hr or less, such as about 27.5 mL/hr or less, such as about 25 mL/hr or less, such as about 22.5 mL/hr or less, such as about 20 mL/hr or less, such as about 17.5 mL/hr or less, such as about 15 mL/hr or less, such as about 12.5 mL/hr or less, such as a rate of delivery of about 10 mL/hr or less.

The OTN catheter assembly of the present disclosure may also generally include a needle. The needle shape and size may be selected based upon the desired target area or based upon the size of catheter desired and the lumen diameter of the catheter. Alternatively, other instruments may be used to pierce a surface of the skin and guide the OTN catheter assembly, such as a tunneler or a guidewire. In a further embodiment, a curved needle may be used, and in such a case a catheter formed from a flexible material capable of holding a shape may be used so as to maintain the shape after a needle is removed from the catheter assembly. A needle of the present disclosure may also be made of an echogenic material, or may include other materials based upon the target area and placement thereof. The needle, the needle tip size, and the needle tip shape may be selected to correspond with a projection of the catheter body of the present disclosure, or a standard sized and shaped needle may be used.

The needle may generally have a length or be inserted into the catheter lumen at a location such that the tip of the needle may extend through the opening in the distal end of the catheter. Only a small portion of the needle may extend through the opening in the distal end or a large portion may extend through the opening in the distal end. In one embodiment, only the tip of the needle may extend through the opening in the distal end such that the catheter is largely in the desired target area when the needle is removed. Alternatively, a longer portion of the needle may extend through the opening in the distal end, and optionally, the catheter's positioning may be adjusted after the needle is removed. The needle may also be configured so as to remain in the lumen in a retracted position such that the needle still allows for the delivery of a drug or anesthesia but is completely contained within the lumen when the projection is in a closed position.

Generally, the OTN catheter assembly of the present disclosure may have a projection that is configured to interact with a needle that may be contained in the catheter lumen such that the projection is in an open position when a needle extends through an opening in the distal end of the catheter, and transitions into a closed position when the needle is withdrawn into the catheter lumen. The projection may close automatically when the needle is withdrawn; for example, the projection may have a treated section (e.g. heat treated) at the point of attachment to the catheter body that transitions the projection into a closed position when the needle is no longer applying pressure or support to the projection. The projection may be releasably or permanently held in the closed position by the portion of the open distal end opposite the projection. This may be accomplished by an additional adhesive, tacky, or textured material on the portion of the open distal end opposite the projection or, alternatively, point of attachment may have a treated section that maintains the projection in a closed position unless acted upon by internal forces such as a needle.

In an embodiment wherein the flap is releasably held in a closed position, a needle may be reinserted through the distal end of the catheter, transitioning the projection into the open position. This may allow a catheter to be repositioned, or may allow the delivery of a bolus or larger single dose of a drug or anesthetic before resuming the continuous flow of the catheter.

In yet a further embodiment, the OTN catheter assembly may also include a needle hub releasably or permanently attached or configured at the proximal end of the catheter. The present disclosure may include both a needle hub and a catheter hub, or may include a singular hub suitable to interact with both a needle and a catheter. Regardless of the type and number of hubs, the hub or hubs may be configured to maintain the needle within the lumen of the catheter until it is desired to remove the needle. Alternatively, the hub may comprise a durable material, such as a rigid plastic in one embodiment, or alternatively a polyethylene polymer or copolymer and/or polypropylene polymer or copolymer, so as to not become disconnected from the catheter and needle when the OTN catheter assembly is placed within a target area. In yet a further embodiment, the hub or hubs may provide a convenient place to hold and/or guide the OTN catheter assembly. Additionally or alternatively, the hub may be provided with ports for administering a drug or anesthesia through the catheter, needle, or both, or may provide a connection to the needle or catheter such as a nerve stimulator device.

The present disclosure may further be generally directed to a method for administering a bolus or continuous flow of a drug or anesthetic to a patient. The method may include piercing a surface of skin with an OTN catheter. The OTN catheter may generally have the form of the OTN catheter assembly as discussed above. After piercing the skin, the distal end of the OTN catheter assembly may be positioned near a target area. The OTN catheter assembly may be fully positioned before withdrawing or removing the needle, or alternatively the OTN catheter assembly may only be partially inserted before withdrawing or removing the needle, and may then be further positioned after withdrawing or removing the needle.

The OTN catheter assembly may further be used to administer a drug or anesthesia to a patient or a target area of a patient. The target area may be a nerve or nerve bundle, or alternatively may be used for other or more general targets. Administration of the drug or anesthesia may begin while the needle is still extended through the opening in the distal end of the catheter or may not begin until the needle has been either withdrawn into the lumen or withdrawn from the lumen such that the projection is in a closed position. The OTN catheter assembly may thus be used to administer a bolus or a continuous flow of a drug or anesthesia. In one embodiment, no drug or anesthesia may be administered through the opening in the distal end and instead is only administered through the at least one aperture. As previously discussed, the shape and size of the catheter, and the shape, size and orientation of the apertures may be selected based upon the amount or type of drug or anesthesia, or based upon the desired rate of delivery, to name a few considerations.

While embodiments of the present disclosure have been generally discussed, various embodiments of the present disclosure will now be described in more detail. Referring now to the drawings, FIGS. 1-11b illustrate various embodiments of the OTN catheter assembly 10 according to the present disclosure. It should be understood that the catheter assembly of the present disclosure may have any suitable catheter assembly configuration known in the art. For example, in certain embodiments, such as FIG. 1, the catheter assembly 10 may have a proximal end 12 and a distal end 14, and a needle 18 may be coaxially located in a lumen 22 (see FIG. 2) of a catheter 16 such that catheter 16 and needle 18 may be simultaneously inserted into a patient. Further, as shown, the catheter 16 has a catheter body 20 that extends in the axial direction A from the proximal end 12 to the distal end 14. The catheter body 20 defines a lumen 22 (see FIGS. 2, 7C, and 7D) in which needle 18 has been inserted. The lumen 22 extends between opposing interior edges 36 of the catheter body 20 and may have a diameter d2 that is larger than a diameter of a needle 18. The catheter body 20 may have a wall thickness t, which may be generally constant from the proximal end 12 to the distal end 14, or which may increase or decrease, where the thickness is defined as the distance between the exterior edge 38 of the catheter body 20 and the interior edge 36 of the catheter body 20. The transverse direction T, generally extends through the catheter 16 perpendicular to the axial direction A.

As shown in FIG. 1, there may be an opening 24 in the distal end 14. As shown in FIG. 1, a needle 18 extends through the opening 24. In addition, the proximal end 12 of the catheter 16 may include a hub 46 configured thereon. The hub 46 may further be configured to interact with both the needle 18 and the catheter 16, or multiple hubs 46 which may be configured to interact with each other may be used such that a hub 46 is used for the catheter 16 and a hub is used for the needle 18 (not shown). Though not shown, a fluid input or delivery device may be attached to the hub 46 or hubs. Though not shown in FIG. 1, it should be understood that any catheter assembly 10 described herein may include at least one aperture for delivery of a drug or anesthesia to a target area or patient.

The OTN catheter assembly, such as illustrated in FIG. 1, may have a projection 26 which may extend in an axial direction A from the distal end 14 of the catheter body 20. The projection 26 is shown in a generally open position in FIG. 1, but the projection 26 may also be in a closed position, or any position between the open and closed position. The projection 26 may be connected to the catheter body 20 by a point of connection 28. Point of connection 28 may be comprised of the same material as the catheter 16 or the projection 26, or may, additionally, or alternatively, be heat treated, chemically treated, mechanically treated or otherwise treated, such that the point of connection 28 may allow the projection 26 to transition, releasably or permanently, into a closed position.

FIG. 2 generally shows the embodiment of FIG. 1 wherein the projection 26 has been transitioned into a closed position. FIG. 2 generally shows a projection that has a length L which is roughly less than the outer diameter d of the catheter and generally greater than the diameter d2 of the lumen, as the portion opposite the projection 30 of the distal end 14 has tapered towards the catheter lumen 22. The embodiment of FIG. 2 generally shows that the projection 26 has transitioned roughly about 45° measured from a starting position of the projection in the first position and a fully closed position which is generally in the plane of the transverse direction T. Therefore, the angle ⊖₁ has been closed between a starting position of the projection 26 in the first position to being aligned with the transverse direction T. The embodiment of FIG. 2 further shows an embodiment wherein the projection 26 forms a generally flat or blunt end 32 when the projection 26 is in a closed position.

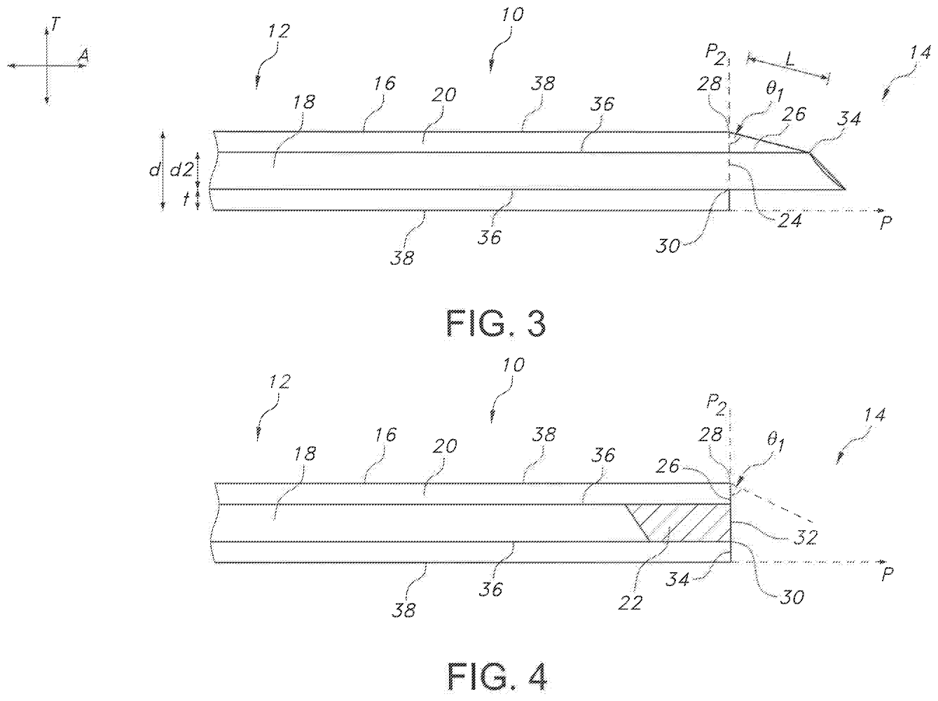

FIGS. 3 and 4 show an additional embodiment of the present disclosure wherein the portion of the distal end opposite the projection 30 has a generally consistent thickness t along the entirety of the catheter body 20. In such an embodiment, the length L of the projection 26 may generally be equal to the outer diameter d of the catheter 16. Alternatively, the length L of the projection 26 may generally be less than or greater than the outer diameter d of the catheter 16 (not shown). In such an embodiment, the tip 34 of the projection 26 may only cover the opening 24 in the distal end 14 so as to roughly meet the interior edge 36 of the catheter body 20, or alternatively, the tip of the projection 34 may overlap and extend past the exterior edge 38 of the catheter body 20. The embodiment, such as shown in FIG. 4, may have a projection 26 which may form a generally flat or blunt end 32 when the projection 26 is in a closed position.

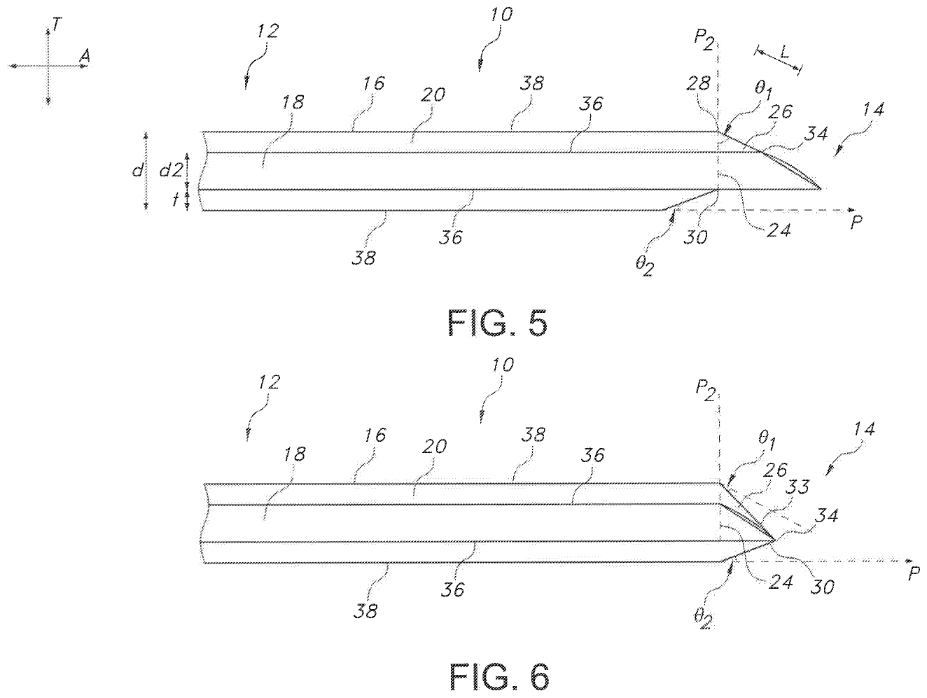

FIGS. 5 and 6 generally show an embodiment of the present disclosure wherein the projection 26 may form a pointed or beveled end 33 when the projection 26 is in a closed position. Such a configuration may comprise a portion of the distal end opposite the projection 30 wherein the end of the portion of the distal end opposite the projection 30 may extend past the plane of the point of connection 28 between the projection 26 and the catheter body 20 as generally portrayed by reference character P₂. As shown generally in FIG. 6, a projection 26 is transitioned into a closed position, and an angle ⊖₁ formed between the projection in the first position and the transverse direction T may be closed, where the angle to be closed may range from about 1° to about 90°. The angle ei to be closed may be less than about 90°, such as less than about 80°, such as less than about 70° such as less than about 65°, such as less than about 60°, such as less than about 55°, such as less than about 50°, such as greater than about 20°, such as greater than about 25°, such as greater than about 30°, such as greater than about 35°, such as greater than about 40°, such as greater than about 45°. As described above, such a projection 26 may have a length L such that the projection meets an interior edge 36 or exterior edge 38 of a catheter body 20 or extends past the exterior edge 38.

FIGS. 7A-7D generally show an OTN catheter assembly of the present disclosure wherein the needle 18 is being withdrawn into the lumen 22. The embodiment shown in FIGS. 7A-7D has a plurality of apertures 40 spaced radially along a surface of the catheter body. Though not shown, the apertures may be spaced axially around the catheter body 20 or may be located nearer to a proximal end 12 instead of a distal end 14. FIGS. 7A-7D show that the projection 26 may transition into a closed position as the needle 18 is withdrawn into the lumen 22. The projection 26 may have a length/such that the tip 34 of the projection 26 meets a portion of the distal end opposite the projection 30 so as to provide an end with a tapered portion or beveled end 33 as well as a blunt end 32. Additionally, FIGS. 7A-7D further show an embodiment where the portion of the distal end opposite the projection 30 has a permanent or releasable attachment portion 42. Such a portion may be an adhesive or a roughened polymer, or a gap or stopper, for example, though the permanent or releasable attachment portion 42 may be attached through other methods or compositions as known in the art. However, the embodiment of FIGS. 7A-7D may have any of the features or arrangements previously discussed.

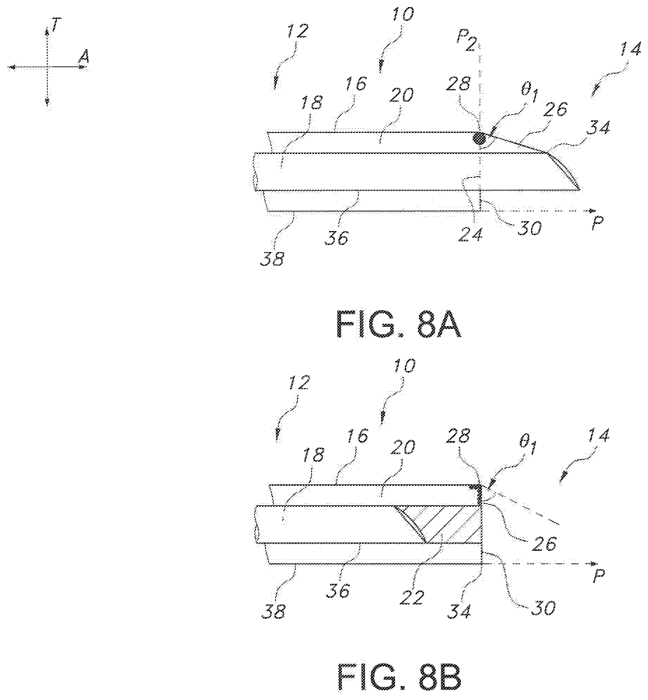

FIGS. 8A-8B generally show a close up view of the point of connection 28 which may be of any of the embodiments of the present disclosure. FIGS. 8A-8B show that the point of connection 28 may be treated, such as mechanically, chemically, by heat, or otherwise such that the point of connection 28 is able to be transitioned from an open position to a closed position or vice versa. The treated portion may be quite small, for example only the point of connection 28 or the portion of the catheter body in the plane P₂ of the point of connection 28. For instance, the heat treated portion may only comprise a section of the catheter body including the point of connection 28 and the immediately adjacent sections such that the treated portion is only about 0.1 mm or less. Alternatively, the treated section may be quite large, such as an area of about 1 mm or greater, such as about 1.5 mm or greater, such as about 2 mm or greater, such as about 2.5 mm or greater, such as about 5 mm or less, such as about 4.5 mm or less, such as about 4 mm or less, around or adjacent to the plane P₂ and point of connection 28. The size or amount of treated portion may be selected based upon factors such as the degree or amount of transition needed to transition into a closed position, the thickness or type of material of the catheter body, as well as other considerations.

FIG. 9 generally shows a view of a distal end of an OTN catheter assembly showing a close up of the catheter body 20 and lumen 22. Additionally as shown in FIG. 9, the catheter body 20 may include a reinforcing or flexible material shown by the cross-hatched section of catheter body 20. Such a material may allow the catheter to withstand repositioning after a needle 18 is withdrawn or removed, or may allow the catheter to be bent into a shape that corresponds to a shape of a needle 18 (not shown). The reinforced section may extend a distance in the axial direction such as about 5 mm or greater from the distal end, such as about 10 mm or greater, such as about 15 mm or greater, such as about 20 mm or greater, such as about 25 mm or greater, such as about 30 mm or greater, such as about 35 mm or greater, such as about 40 mm or greater, such as about 45 mm or greater, such as about 50 mm or greater, such as about 500 mm or less, such as about 450 mm or less, such as about 400 mm or less, such as about 350 mm or less, such as about 300 mm or less, such as about 250 mm or less, such as about 200 mm or less, such as about 150 mm or less, such as about 100 mm or less from the distal end, or alternatively, the entirety of the catheter body 20 may be treated. Though not shown, the reinforced section may also include at least one aperture 40. Alternatively, the apertures 40 may only be located on a section of the catheter body 20 that does not have a reinforcing material. In yet a further embodiment, the apertures 40 may be located in both reinforced areas and unreinforced areas. If used, a flexible material may extend in a similar area as the reinforced section or may alternatively be included along the entire length of the catheter 16.

FIGS. 10A and 10B show cross sections taken proximal from the tip of OTN catheter assemblies of the present disclosure. FIGS. 10A-10B show a catheter body 20 defining a lumen 22, wherein a needle 18 is disposed within the catheter lumen 22. As shown, the diameter of the needle 18 is smaller than a diameter of the lumen defined by the interior edge of the catheter body 36. Apertures 40 may be disposed axially around the catheter body 20 as shown in 10B or may be located generally in a single axial plane such as shown in FIG. 10A.

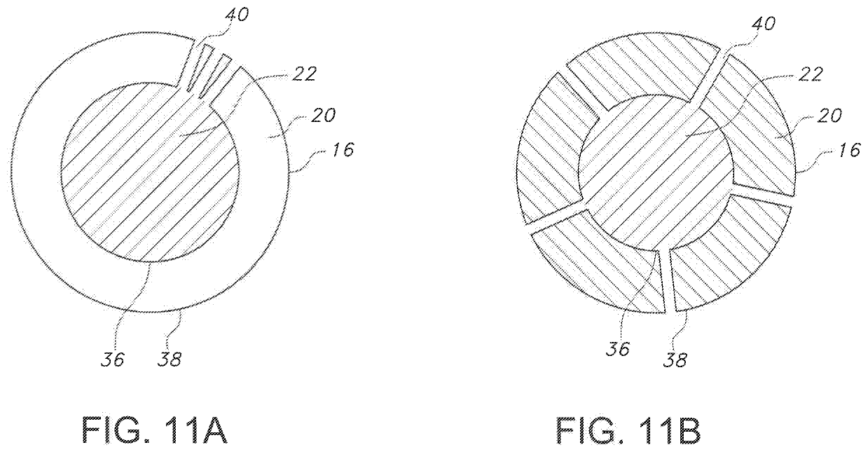

FIGS. 11A and 11B show cross sections taken proximal from the tip, similar to FIGS. 10A and 10B, except that in the present embodiment, the needle 18 has been withdrawn from this portion of the lumen 22. Apertures 40 may be disposed axially around the catheter body 20 as shown in 11B or may be located generally in a single axial plane such as shown in FIG. 11A.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. An over the needle catheter assembly comprising, a catheter having a proximal end and a distal end, a body that extends from the proximal end to the distal end in an axial direction, wherein the body defines a lumen extending from the proximal end to the distal end, the body having at least one aperture and a projection that extends axially from the distal end of the body when the projection is in an open position, the projection being sized and connected to the body such that the projection closes an opening in the distal end of the catheter when the projection is transitioned into a closed position; and
a needle releasably configured within the lumen of the catheter, wherein the needle extends beyond the distal end of the catheter in a first position and is withdrawn into the lumen or is removed from the catheter in a second position.

2. The over the needle catheter assembly of claim 1, further comprising a hub, the hub being fixedly or releasably secured to the proximal end of the catheter.

3. The over the needle catheter assembly of claim 1, wherein the projection is attached to the body via a heat treatment, a mechanical treatment, a chemical treatment, an adhesive or combinations thereof.

4. The over the needle catheter assembly of claim 1, wherein the projection has an axial length from a point of connection with the body to a distal end of the projection that is at least as long as an outer diameter of the catheter.

5. The over the needle catheter assembly of claim 1, wherein the projection is configured to transition into the closed position when the needle is withdrawn into the lumen.

6. The over the needle catheter assembly of claim 1, wherein the projection is configured to close the opening in the distal end such that the distal end of the catheter is beveled, pointed, rounded, flat, or blunt when the projection is in the closed position.

7. The over the needle catheter assembly of claim 1, comprising at least two apertures.

8. The over the needle catheter assembly of claim 7, wherein the at least two apertures are axially spaced apart along the body of the catheter.

9. The over the needle catheter assembly of claim 7, wherein the at least two apertures are radially spaced apart around the body of the catheter.

10. The over the needle catheter assembly of claim 7, wherein each aperture has a diameter from about 100 micrometers to about 1 millimeter.

11. The over the needle catheter assembly of claim 1, wherein the catheter is comprised of a polymer, wherein the polymer comprises a polyester, a polyurethane, a polyamide, a polyolefin, or a combination thereof.

12. The over the needle catheter assembly of claim 11, wherein the polymer further comprises a reinforcing material or a flexible material.

13. The over the needle catheter assembly of claim 1, wherein a portion of the body opposite the projection is configured to releasably or permanently attach to the projection when the projection is in the closed position.

14. An over the needle catheter assembly comprising,
a catheter having a proximal end and a distal end, a body that extends from the proximal end to the distal end in an axial direction, wherein the body defines a lumen extending from the proximal end to the distal end, the body having at least one aperture and a projection that extends axially from the distal end of the body when the projection is in an open position, the projection being sized and connected to the body such that the projection closes an opening in the distal end of the catheter when the projection is transitioned into a closed position;
wherein the lumen has a diameter larger than a diameter of a needle, wherein the needle extends beyond the distal end of the catheter when the needle is fully inserted through the opening in the distal end of the catheter, the projection being connected to the body such that the projection is in the open position when a needle is fully inserted through the opening in the distal end of the catheter and transitions into the closed position when a needle is retracted from the lumen such that the needle does not extend past the distal end of the catheter.

15. A method for administering a bolus or continuous flow of a drug or anesthetic to a target area of a patient comprising:
piercing a surface of skin of the patient with an over the needle catheter assembly, the over the needle catheter assembly comprising a catheter having a proximal end and a distal end; a body that extends from the proximal end to the distal end in an axial direction, wherein the body defines a lumen extending from the proximal end to the distal end, the body having at least one aperture and a projection that extends axially from the distal end of the body when the projection is in an open position, the projection being sized and connected to the body such that the projection closes an opening in the distal end of the catheter when the projection is transitioned into a closed position; and a needle releasably configured within the lumen of the catheter, wherein the needle extends beyond the distal end of the catheter in a first position and is withdrawn into the lumen or is removed from the catheter in a second position;
positioning the distal end of the over the needle catheter assembly adjacent the target area of the patient;
withdrawing the needle into the lumen at least to a point of connection between the body and the projection or withdrawing the needle completely such that the projection transitions into the closed position; and
administering a bolus or continuous flow of a drug or anesthetic to the target area.

16. The method of claim 15, wherein the catheter is repositioned after the needle has been at least partially withdrawn.

17. The method of claim 15, wherein the over the needle catheter assembly is positioned near a nerve or nerve bundle.

18. The method of claim 15, wherein the needle is withdrawn prior to reaching the target area.

19. The method of claim 15, wherein the at least one aperture is sized such that a drug or anesthetic is administered at a rate of about 0.01 ml/hr to about 35 mL/hr.

20. The method of claim 15, wherein the opening in the distal end of the catheter is completely closed by the projection such that no drug or anesthetic is administered from the opening in the distal end of the catheter and a drug or anesthetic is only administered from the at least one aperture.
